(19) United States
(12) Patent Application Publication
(10) Pub. No.: US 2020/0166674 A1
(43) Pub. Date: May 28, 2020

(54) ANTI-FOG LENS

(71) Applicant: Chih Ming Wu, TAINAN (TW)

(72) Inventor: Chih Ming Wu, TAINAN (TW)

(73) Assignee: KODA OPTICAL CO., LTD., TAINAN (TW)

(21) Appl. No.: 16/198,787

(22) Filed: Nov. 22, 2018

(51) Int. Cl. G02B 1/18; A61F 9/02; G02C 7/02

(52) U.S. Cl. CPC G02B 1/18; G02C 7/02; A61F 9/028

(57) ABSTRACT

An anti-fog lens includes an optical body, made of polycarbonates, and an anti-fog film combination located at a rear surface of the optical body. The anti-fog film combination is constructed of an anti-fog film made of triacetate, and a polycarbonate film, which are joined together by spreading an optical adhesive therebetween. The optical body and the anti-fog film combination are subsequently placed in a mold into which a molten polycarbonate material is fed. As such, the optical body and the anti-fog film can be joined together after the molten polycarbonate material is cooled and solidified. Preferably, the anti-fog film has a thickness between 0.3 and 0.6 mm, and the polycarbonate film has a thickness between 0.3 and 0.6 mm. The anti-fog lens can achieve a stable and excellent performance on prevention of fog.

### BACKGROUND OF THE INVENTION

### 1. Field of the Invention

The present invention relates to an anti-fog lens and, more particularly, to a lens which includes an anti-fog film pre-attached to a polycarbonate film and subsequently placed in a mold to be securely attached on an optical body under a high temperature, so that the lens can achieve a stable and excellent performance on prevention of fog.

### 2. Description of the Prior Art

For a general lens made of a polycarbonate material, due to the confined space of the associated goggles and the body temperature of a wearer's body, fogging may happen on the lens. To achieve anti-fog effect, it is necessary to take corresponding anti-fog measures for a lens. The most common measure is using an anti-fog film, which can be attached to a rear surface of a lens via an adhesive. However, this attachment is insecure. For polycarbonate lenses, due to their smooth surfaces, they are somewhat difficult to be attached with an anti-fog film, such as a triacetate (TAC) film. Even though a polycarbonate lens is configured with recesses thereon to facilitate attachment with an anti-fog film, a secure bonding effect cannot be ensured. Thus, there is a need for improvement.

### SUMMARY OF THE INVENTION

One object of the present invention is to provide an anti-fog lens, which has a stable and excellent performance on prevention of fog.

The anti-fog lens comprises an optical body made of polycarbonates and an anti-fog film combination located at a rear surface of the optical body. The anti-fog film combination is constructed of an anti-fog film made of triacetate, and a polycarbonate film, which are joined together by spreading an optical adhesive therebetween. The optical body and the anti-fog film combination are placed in a mold into which a molten polycarbonate material is injected. As such, the optical body and the anti-fog film can be joined together after the molten polycarbonate material is cooled and solidified. Preferably, the anti-fog film has a thickness between 0.3 and 0.6 mm, and the polycarbonate film has a thickness between 0.3 and 0.6 mm.

One advantageous feature of the anti-fog lens is that an anti-fog film associated therewith is pre-attached to a polycarbonate film to form an anti-fog film combination, which is subsequently placed on an optical body located in a mold, which is subsequently filled with a molten polycarbonate material, whereby the high temperature of the molten polycarbonate material enables the optical body and the anti-fog film to be joined securely.

Other objects, advantages, and novel features of the present invention will become more apparent from the following detailed description when taken in conjunction with the accompanying drawings.

### BRIEF DESCRIPTION OF DRAWINGS

FIG. 1 shows a 3-dimensional view of an anti-fog lens according to one embodiment of the present invention.

FIG. 2 shows an exploded view of the anti-fog lens.

FIG. 3 shows a 3-dimension view of an anti-fog lens, wherein an optical body thereof is provided with adjustment elements.

FIG. 4 shows an exploded view of the anti-fog lens provided with the adjustment elements.

FIG. 5 shows a 3-dimensional view of an anti-fog lens, which is configured for use in snow goggles.

FIG. 6 shows a 3-dimensional view of an anti-fog lens, which is configured for use in another snow goggles.

### DETAILED DESCRIPTION OF THE PREFERRED EMBODIMENTS

To illustrate the features and effect of the present invention, a preferred embodiment with reference to the accompanying drawings is provided in the following.

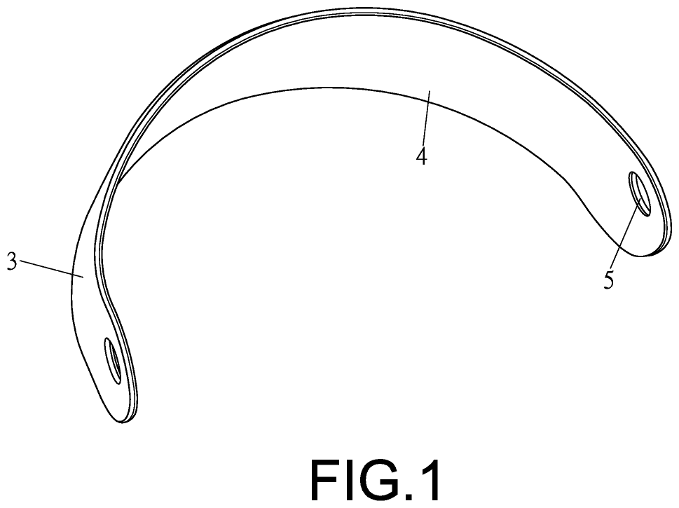

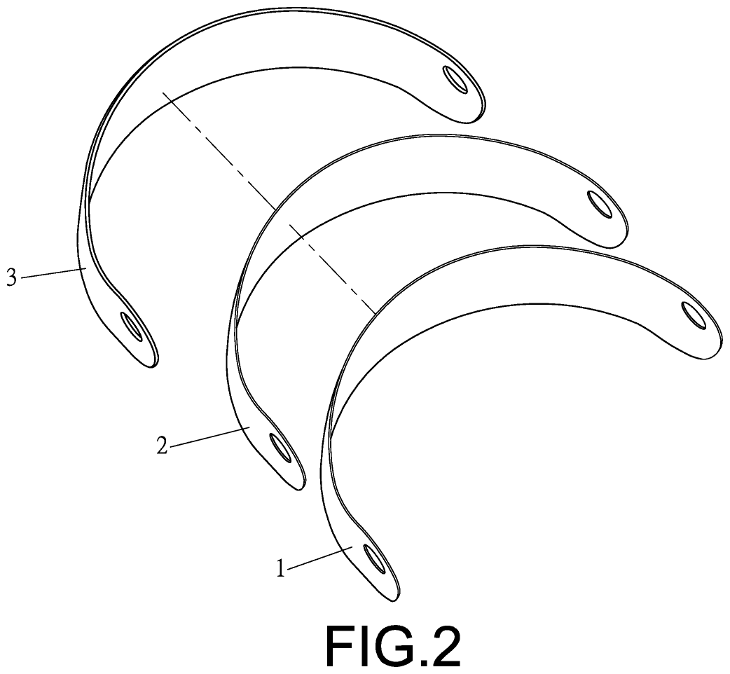

Referring to FIGS. 1 and 2, an anti-fog lens according to one embodiment of the present invention is shown, which generally comprises an optical body (transparent body) 3 made of polycarbonates, and an anti-fog film combination 4 located at a rear surface of the optical body 3. The anti-fog film combination 4 is constructed of an anti-fog film 1, which can be made of triacetate (TAC), and a polycarbonate (PC) film 2, which are joined together by spreading an optical adhesive therebetween to become a three-layer structure, wherein the anti-fog film 4 is arranged on a rear surface of the polycarbonate film 2. Preferably, the anti-fog film 1 has a thickness between 0.3 and 0.6 mm, and the polycarbonate film 2 also has a thickness between 0.3 and 0.6 mm.

The anti-fog lens of the present invention can be configured to any shapes and applied to various helmets and goggles. As an application example shown in FIGS. 1 and 2, the anti-fog lens of the present invention can be employed as a visor of a helment, wherein the anti-fog film combination 4 is sized to match with the polycarbonate optical body 3. Also, two mounting holes 5 are defined at two sides of the lens and extend through the optical body 3 and the anti-fog film combination 4.

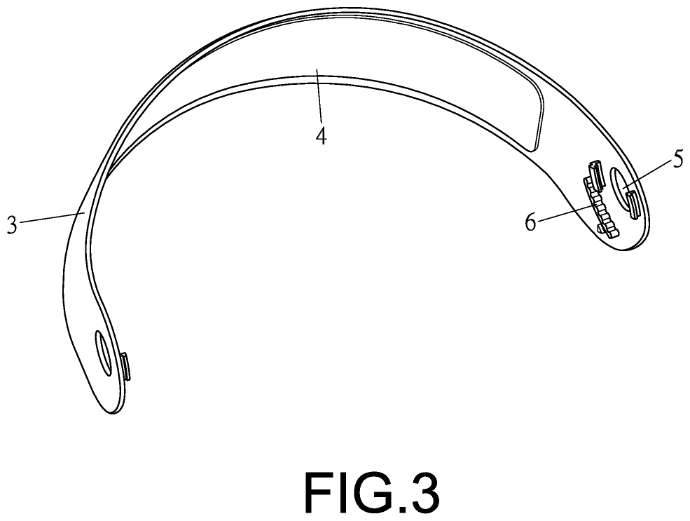

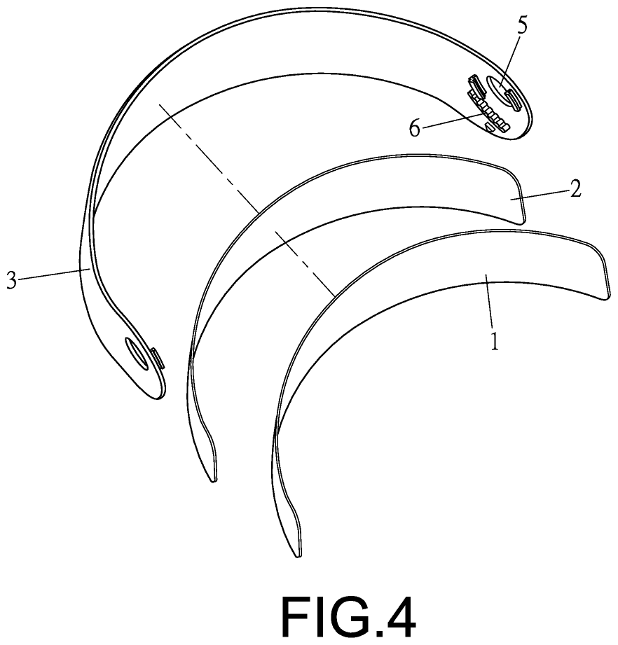

As another application example shown in FIGS. 3 and 4, the optical body 3 defines two mounting holes 5 and are provided with two adjustment elements 6 adjacent to the mounting holes 5. The anti-fog film combination 4 is smaller than the optical body 3 and joined to an intermediate portion of the optical body 3.

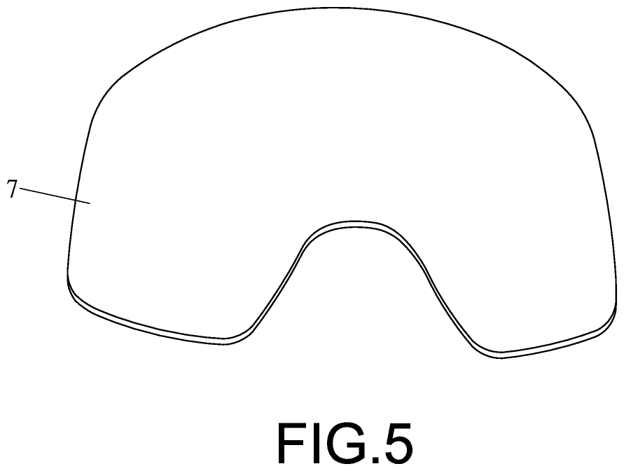

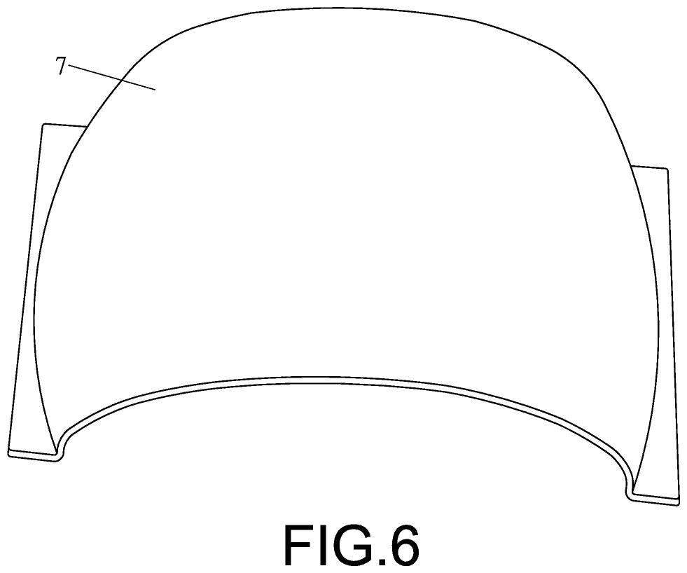

As a further application example shown in FIGS. 5 and 6, the anti-fog lens of the present invention is configured as a snow goggle lens.

A method for manufacturing the anti-fog lens of the present invention may include the following steps:

Step 1: joining an anti-fog film 1 and a polycarbonate film 2 together by spreading an optical adhesive therebetween to form an anti-fog film combination 4, which can be further shaped by heat, wherein the anti-fog film 1 is arranged on a rear surface of the polycarbonate film 2;

Step 2: placing the anti-fog film combination 4 in a mold, on a rear surface of an optical body 3 that had been located in the mold; and

Step 3: injecting a molten polycarbonate material into the mold such that the interior of the mold achieves a temperature between 260 and 280 degrees C. upon the molten polycarbonate material being fed into the mold, thus forming a multi-layer anti-fog lens after the molten polycarbonate material being cooled and solidified.

It is noted that the above temperature range, between 260 and 280 degrees C., is the temperature of the polycarbonate material which has been fully melted and fed by a screw to enter the mold. There is no limitation about the pressure of the molten polycarbonate material and the time of the material retained in the mold, if the two pieces can be secured. Also, it is noted that the polycarbonate film 2 is indispensable in making an anti-fog lens. Thus, the polycarbonate film 2 serves as a key element in the present invention. With the polycarbonate film 2 pre-attached with the anti-fog film 1 to form a combination, an anti-fog lens can be successfully produced in the subsequent molding process.

While the invention has been described with reference to the preferred embodiment above, it should be recognized that the preferred embodiment is given for the purpose of illustration only and is not intended to limit the scope of the present invention and that various modifications and changes, which will be apparent to those skilled in the relevant art, may be made without departing from the scope of the invention.

What is claimed is:

1. An anti-fog lens, which comprises an optical body made of polycarbonates and an anti-fog film combination located at a rear surface of the optical body, wherein the anti-fog film combination is constructed of an anti-fog film and a polycarbonate film joined together by spreading an optical adhesive therebetween; the optical body and the anti-fog film combination are subsequently placed in a mold into which a molten polycarbonate material is fed, whereby the optical body and the anti-fog film are joined together after the molten polycarbonate material is cooled and solidified.

2. The anti-fog lens of claim 1, wherein the anti-fog film is made of triacetate.

3. The anti-fog lens of claim 2, wherein the anti-fog film has a thickness between 0.3 and 0.6 mm; the polycarbonate film has a thickness between 0.3 and 0.6 mm.

4. The anti-fog lens of claim 3, wherein the temperature inside the mold reaches about 260 to 280 degrees C. upon the molten polycarbonate material being fed into the mold.
